(19) United States
(12) Patent Application Publication
(10) Pub. No.: US 2011/0220364 A1
(43) Pub. Date: Sep. 15, 2011

(54) PROCESS FOR MINERAL OIL PRODUCTION USING CATIONIC SURFACTANTS HAVING A HYDROPHOBIC BLOCK WITH A CHAIN LENGTH OF 6 TO 10 CARBON ATOMS

(75) Inventors: CHRISTIAN BITTNER, Bensheim (DE); Günter Oetter, Frankenthal (DE); Jack Tinsley, Mannheim (DE); Christian Spindler, Ludwigshafen (DE); Gabriela Alvarez Jürgenson, Mannheim (DE); Sophie Vogel, Mannheim (DE)

(73) Assignee: BASF SE, Ludwigshafgen (DE)

(21) Appl. No.: 13/043,210

(22) Filed: Mar. 8, 2011

Related U.S. Application Data
(60) Provisional application No. 61/312,302, filed on Mar. 10, 2010.

(51) Int. Cl. E21B 43/00; C07C 211/00

(52) U.S. Cl. 166/369; 564/463

(57) ABSTRACT

The invention relates to a process for mineral oil production by means of Winsor type III microemulsion flooding, in which an aqueous surfactant formulation comprising at least one ionic surfactant of the general formula R¹N⁺(R²)ₘ(R³)ₙ(R⁴)X⁻ is injected through injection boreholes into a mineral oil deposit, and crude oil is withdrawn from the deposit through production boreholes.

The invention relates to a process for mineral oil production by means of Winsor type III microemulsion flooding, in which an aqueous surfactant formulation comprising at least one ionic surfactant of the general formula

R¹N⁺(R²)ₘ(R³)ₙ(R⁴)X⁻

is injected through injection boreholes into a mineral oil deposit, and crude oil is withdrawn from the deposit through production boreholes.

The invention further relates to ionic surfactants according to the general formula and to processes for preparation thereof.

In natural mineral oil deposits, mineral oil is present in the cavities of porous reservoir rocks which are sealed toward the surface of the earth by impervious top layers. The cavities may be very fine cavities, capillaries, pores or the like. Fine pore necks may, for example, have a diameter of only about 1 μm. As well as mineral oil, including fractions of natural gas, a deposit comprises water with a greater or lesser salt content.

In mineral oil production, a distinction is generally drawn between primary, secondary and tertiary production. In primary production, the mineral oil flows, after commencement of drilling of the deposit, of its own accord through the borehole to the surface owing to the autogenous pressure of the deposit.

After primary production, secondary production is therefore used. In secondary production, in addition to the boreholes which serve for the production of the mineral oil, the so-called production bores, further boreholes are drilled into the mineral oil-bearing formation. Water is injected into the deposit through these so-called injection bores in order to maintain the pressure or to increase it again. As a result of the injection of the water, the mineral oil is forced slowly through the cavities into the formation, proceeding from the injection bore in the direction of the production bore. However, this only works for as long as the cavities are completely filled with oil and the more viscous oil is pushed onward by the water. As soon as the mobile water breaks through cavities, it flows on the path of least resistance from this time, i.e. through the channel formed, and no longer pushes the oil onward.

By means of primary and secondary production, generally only approx. 30 to 35% of the amount of mineral oil present in the deposit can be produced.

It is known that the mineral oil yield can be enhanced further by measures for tertiary oil production. A review of tertiary oil production can be found, for example, in “Journal of Petroleum Science of Engineering 19 (1998)”, pages 265 to 280. Tertiary oil production includes, for example, thermal methods in which hot water or steam is injected into the deposit. This lowers the viscosity of the oil. The flow medium used may likewise be gases such as CO₂ or nitrogen.

Tertiary mineral oil production also includes methods in which suitable chemicals are used as assistants for oil production. These can be used to influence the situation toward the end of the water flow and as a result also to produce mineral oil hitherto held firmly within the rock formation.

Viscous and capillary forces act on the mineral oil which is trapped in the pores of the deposit rock toward the end of the secondary production, the ratio of these two forces relative to one another being determined by the microscopic oil separation. By means of a dimensionless parameter, the so-called capillary number, the action of these forces is described. It is the ratio of the viscosity forces (velocity×viscosity of the forcing phase) to the capillary forces (interfacial tension between oil and water×wetting of the rock):

$${N}_{c} = \frac{μ v}{σcos θ} .$$

In this formula, μ is the viscosity of the fluid mobilizing mineral oil, ν is the Darcy velocity (flow per unit area), σ is the interfacial tension between liquid mobilizing mineral oil and mineral oil, and θ is the contact angle between mineral oil and the rock (C. Melrose, C. F. Brandner, J. Canadian Petr. Techn. 58, October-December, 1974). The higher the capillary number, the greater the mobilization of the oil and hence also the degree of oil removal.

It is known that the capillary number toward the end of secondary mineral oil production is in the region of about 10⁻⁶ and that it is necessary to increase the capillary number to about 10⁻³ to 10⁻² in order to be able to mobilize additional mineral oil.

For this purpose, it is possible to conduct a particular form of the flooding method—what is known as Winsor type III microemulsion flooding. In microemulsion flooding, the injected surfactants should form a Winsor type III microemulsion with the water phase and oil phase present in the deposit. A Winsor type III microemulsion is not an emulsion with particularly small droplets, but rather a thermodynamically stable, liquid mixture of water, oil and surfactants. The three advantages thereof are that
a very low interfacial tension σ between mineral oil and aqueous phase is thus achieved,
it generally has a very low viscosity and as a result is not trapped in a porous matrix,
it forms with even the smallest energy inputs and can remain stable over an infinitely long period (conventional emulsions, in contrast, require high shear forces which predominantly do not occur in the reservoir, and are merely kinetically stabilized).

The Winsor type III microemulsion is in an equilibrium with excess water and excess oil. Under these conditions of microemulsion formation, the surfactants cover the oil-water interface and lower the interfacial tension σ more preferably to values of <10⁻² mN/m (ultra-low interfacial tension). In order to achieve an optimal result, the proportion of the microemulsion in the water-microemulsion-oil system, with a defined amount of surfactant, should by its nature be at a maximum, since correspondingly lower interfacial tensions can be achieved.

In this manner, it is possible to alter the form of the oil droplets (interfacial tension between oil and water is lowered to such a degree that the smallest interface state is no longer favored and the spherical form is no longer preferred), and they can be forced through the capillary openings by the flooding water.

When all oil-water interfaces are covered with surfactant, in the presence of an excess amount of surfactant, the Winsor type III microemulsion forms. It thus constitutes a reservoir for surfactants which cause a very low interfacial tension between oil phase and water phase. By virtue of the Winsor type III microemulsion being of low viscosity, it also migrates through the porous deposit rock in the flooding process (emulsions, in contrast, can become trapped in the porous matrix and block deposits). When the Winsor type III microemulsion meets an oil-water interface as yet uncovered with surfactant, the surfactant from the microemulsion can significantly lower the interfacial tension of this new interface, and lead to mobilization of the oil (for example by deformation of the oil droplets).

The oil droplets can subsequently combine to a continuous oil bank. This has two advantages:

Firstly, as the continuous oil bank advances through new porous rock, the oil droplets present there can coalesce with the bank.

Moreover, the combination of the oil droplets to give an oil bank significantly reduces the oil-water interface and hence surfactant no longer required is released again. Thereafter, the surfactant released, as described above, can mobilize oil droplets remaining in the formation.

Microemulsion flooding is consequently an exceptionally efficient process, and requires much less surfactant compared to an emulsion flooding process. In microemulsion flooding, the surfactants are typically optionally injected together with co-solvents and/or basic salts (optionally in the presence of chelating agents). Subsequently, a solution of thickened polymer is injected for mobility control. A further variant is the injection of a mixture of thickening polymer and surfactants, co-solvents and/or basic salts (optionally with chelating agent), and then a solution of thickening polymer for mobility control. These solutions should generally be clear in order to prevent blockages of the reservoir.

The requirements on surfactants for tertiary mineral oil production differ significantly from requirements on surfactants for other applications: suitable surfactants for tertiary oil production should reduce the interfacial tension between water and oil (typically approx. 20 mN/m) to particularly low values of less than 10⁻² mN/m in order to enable sufficient mobilization of the mineral oil. This has to be done at the customary deposit temperatures of approx. 15° C. to 130° C. and in the presence of water of high salt contents, more particularly also in the presence of high proportions of calcium and/or magnesium ions; the surfactants thus also have to be soluble in deposit water with a high salt content.

To fulfill these requirements, there have already been frequent proposals of mixtures of surfactants, especially mixtures of anionic and nonionic surfactants.

U.S. Pat. No. 4,374,734 discloses the use of cationic surfactants as demulsifiers for breaking emulsions in mineral oil production. One example mentioned is dioctyldimethyl-ammonium chloride.

U.S. Pat. No. 4,596,662 discloses a combination of 30 to 70% glycol diester of a sulfosuccinate, 30 to 50% of a propoxylated alkylamine and 0.1 to 4% of an alkylphenol ether sulfate. The propoxylated alkylamine may comprise 2 to 20 PO units, and also alkyl radicals having 1 to 6 carbon atoms on the nitrogen.

DD 260 713 A1 discloses the formation of microemulsions in the case of simultaneous use of a C₁₂-C₁₈-alkylsulfonate sodium salt and a C₁₂-C₁₈-alkyldimethylbenzyl-ammonium chloride.

WO 93/04265 A1 discloses a mixture of one anionic and one cationic surfactant, which is said to exhibit no precipitate formation on combination of the surfactants and good foaming performance. The cationic surfactant is a dodecyl(bishydroxymethyl)methyl-ammonium chloride.

The use parameters, for example type, concentration and mixing ratio of the surfactants used with respect to one another, are therefore adjusted by the person skilled in the art according to the conditions existing in a given oil formation (for example temperature and salt content).

As described above, mineral oil production is proportional to the capillary number. The lower the interfacial tension between oil and water, the higher the capillary number. Low interfacial tensions with simultaneously sufficient solubility of the surfactant are usually difficult to achieve. This is the case especially when no basic salts which convert carboxylic acids present in the crude oil to hydrophobic surfactants are added (in which case it would merely have been necessary to inject a hydrophilic surfactant which thus has good water solubility). Combinations of long-chain cationic surfactants and long-chain anionic surfactants can precipitate out as an uncharged complex or be lost through dissolution in the oil in the case of an unfavorable combination. Use of cationic counterions, for example tetraethylammonium, with anionic surfactants is unlikely to be successful owing to the high salinities, since there is a high excess of sodium ions. In the course of the flooding process, an exchange of the counterions would take place.

It is therefore an object of the invention to provide a particularly suitable surfactant for use for surfactant flooding or for preferred microemulsion flooding, and an improved process for tertiary mineral oil production.

Accordingly, a process is provided for tertiary mineral oil production by means of Winsor type III microemulsion flooding, in which an aqueous surfactant formulation comprising at least one ionic surfactant, for the purpose of lowering the interfacial tension between oil and water to <0.1 mN/m, preferably to <0.05 mN/m, more preferably to <0.01 mN/m, is injected through at least one injection borehole into a mineral oil deposit, and crude oil is withdrawn from the deposit through at least one production borehole, wherein the surfactant formulation comprises at least one surfactant of the general formula

R¹N⁺(R²)ₘ(R³)ₙ(R⁴)X⁻ where
R¹ is a linear or branched, saturated or unsaturated, aliphatic and/or aromatic hydrocarbyl radical having 6 to 10 carbon atoms,
R² and R³ are each independently ethyleneoxy, propyleneoxy and/or butyleneoxy and/or pentyleneoxy, preferably ethyleneoxy and/or propyleneoxy and more preferably ethyleneoxy,
R⁴ is an alkyl group or hydroxyalkyl group having 1 to 4 carbon atoms, a benzyl group, or a phenyl-CH₂—CH₂— or a phenyl-CH(CH₃)-group,
m is from 1 to 8, and
n is from 1 to 8, where the sum of m+n is in the range from 2 to 8, and
X is an anion.

In a further preferred embodiment of the above-described process, the surfactant formulation comprises at least one surfactant of the general formula

R¹N⁺(R²)ₘ(R³)ₙ(R⁴)X⁻ where
R¹ is a linear or branched, saturated or unsaturated, aliphatic and/or aromatic hydrocarbyl radical having 6 to 10 carbon atoms,
R² and R³ are each independently methyl radicals, ethyl radicals and/or benzyl radicals,
R⁴ is an alkyl group or hydroxyalkyl group having 1 to 4 carbon atoms, a benzyl group, or a phenyl-CH₂—CH₂— or a phenyl-CH(CH₃)-group,
n=m=1, and
X is an anion.

Additionally provided has been a surfactant mixture for mineral oil production, which comprises at least one ionic surfactant of the above-defined general formulae.

With regard to the invention, the following should be stated specifically:

In the process according to the invention for tertiary mineral oil production by means of Winsor type III microemulsion flooding, the use of the inventive surfactant lowers the interfacial tension between oil and water to values of <0.1 mN/m, preferably to <0.05 mN/m, more preferably to <0.01 mN/m. The interfacial tension between oil and water is thus lowered to values in the range from 0.1 mN/m to 0.0001 mN/m, preferably to values in the range from 0.05 mN/m to 0.0001 mN/m, more preferably to values in the range from 0.01 mN/m to 0.0001 mN/m.

In the above-described process according to the invention for mineral oil production, an aqueous surfactant formulation is used, which comprises at least one surfactant of the general formula. It may additionally comprise further surfactants and/or other components.

The at least one surfactant can be summarized by the general formula R¹N⁺(R²)ₘ(R³)ₙ(R⁴)X⁻ as defined above. As a result of production, it is also possible for a plurality of different surfactants which can be summarized by the general formula to be present in the surfactant formulation.

The R¹ radical is a linear or branched, saturated or unsaturated, aliphatic and/or aromatic hydrocarbyl radical having 6 to 10 carbon atoms. In a preferred embodiment of the invention, the R¹ radical is a hexyl radical, octyl radical, 2-ethylhexyl radical, isononyl radical, decyl radical or 2-propylheptyl radical.

In the above general formula, R² and R³ are each independently defined as methyl, ethyl or benzyl, or are each ethyleneoxy, propyleneoxy and/or butyleneoxy and/or pentyleneoxy. The ethyleneoxy, propyleneoxy and butyleneoxy group(s) and pentyleneoxy group(s) are in random distribution, alternating distribution, or are in the form of two, three, four or more blocks in any desired sequence.

In the above-defined general formula, m and n are each integers. However, it is clear to the person skilled in the art in the field of the polyalkoxylates that this definition is the definition of a single surfactant in each case. In the case of presence of surfactant mixtures or surfactant formulations which comprise several surfactants of the general formula, the numbers m and n are averages over all molecules of the surfactants, since the alkoxylation of amines with ethylene oxide or propylene oxide or butylene oxide or pentylene oxide always gives a certain distribution of chain lengths. This distribution can be described in a manner known in principle by what is called the polydispersity D. D=M_{w}/Mₙ is the quotient of the weight-average molar mass and the number-average molar mass. The polydispersity can be determined by means of methods known to those skilled in the art, for example by means of gel permeation chromatography.

In the above general formula, m is from 1 to 8, preferably from 1 to 4.

In the above general formula, n is from 1 to 8, preferably from 1 to 4.

According to the invention, the sum of m+n is a number in the range from 2 to 8, preferably in the range from 2 to 5.

In the above-defined general formula, R⁴ is an alkyl group or a hydroxyalkyl group having 1 to 4 carbon atoms. In a further embodiment of the invention, R⁴ is a benzyl group, or a phenyl-CH₂—CH₂— or a phenyl-CH(CH₃)— group. In a preferred embodiment of the invention, R⁴ is selected from the group of the methyl, ethyl, propyl and butyl groups.

“Hydroxyalkyl” means an alkyl group substituted by one hydroxyl group. Hydroxy-lower alkyl groups are preferred. Examples of preferred groups include: hydroxymethyl, 2-hydroxyethyl, 2-hydroxypropyl, 3-hydroxypropyl, and 2-hydroxybutyl.

In the above formula, X⁻ is an anion, preferably an anion selected from the group of chloride, bromide, iodide, sulfate, methylsulfonate, methosulfate, carbonate and phosphate.

The surfactants of the general formula can be prepared in a manner known in principle by alkoxylating corresponding primary amine. The performance of such alkoxylations is known in principle to those skilled in the art. It is likewise known to the person skilled in the art that the reaction conditions, especially the selection of the catalyst, can influence the molar mass distribution of the alkoxylates.

### Further Surfactants

In addition to the surfactants of the general formula, the formulation may additionally optionally comprise further surfactants. Mention should be made here, for example, of anionic surfactants without alkoxy groups, such as alkylbenzenesulfonates, olefin-sulfonates, paraffinsulfonates, alkyl carboxylates, alkyl sulfates and/or alkyl phosphates, anionic surfactants with alkoxy groups, such as ether sulfates (more preferably alkyl propoxy sulfates), ether sulfonates, ether carboxylates and ether phosphates; alkyl alkoxylates such as alkyl ethoxylates, alkylpropoxy ethoxylates, or else betaine or zwitterionic surfactants such as alkyldimethylamine oxides. These further surfactants may especially also be oligomeric or polymeric surfactants. It is advantageous to use such co-surfactants to reduce the amount of surfactants needed to form a microemulsion.

Such polymeric co-surfactants are therefore also referred to as “microemulsion boosters”. Examples of such polymeric surfactants comprise amphiphilic block copolymers which comprise at least one hydrophilic block and at least one hydrophobic block. Examples comprise polypropylene oxide-polyethylene oxide block copolymers, polyisobutene-polyethylene oxide block copolymers, and comb polymers with polyethylene oxide side chains and a hydrophobic main chain, where the main chain preferably comprises essentially olefins or (meth)acrylates as monomers. The term “polyethylene oxide” here should in each case include polyethylene oxide blocks comprising propylene oxide units as defined above. Further details of such surfactants are disclosed in WO 2006/131541 A1.

### Process for Mineral Oil Production

In the process according to the invention for mineral oil production by means of Winsor type III microemulsion flooding, a suitable aqueous formulation of the surfactants of the general formula is injected through at least one injection borehole into the mineral oil deposit, and crude oil is withdrawn from the deposit through at least one production borehole. The term “crude oil” in this context of course does not mean single-phase oil, but rather the usual crude oil-water emulsions. In general, a deposit is provided with several injection boreholes and with several production boreholes. The main effect of the surfactant lies in the reduction of the interfacial tension between water and oil—desirably to values significantly <0.1 mN/m. After the injection of the surfactant formulation, known as “surfactant flooding”, or preferably the microemulsion flooding, the pressure can be maintained by injecting water into the formation (“water flooding”) or preferably a higher-viscosity aqueous solution of a polymer with strong thickening action (“polymer flooding”). Also known, however, are techniques by which the surfactants are first of all allowed to act on the formation. A further known technique is the injection of a solution of surfactants and thickening polymers followed by a solution of thickening polymer. The person skilled in the art is aware of details of the industrial performance of “surfactant flooding”, “water flooding”, and “polymer flooding”, and employs an appropriate technique according to the type of deposit.

For the process according to the invention, an aqueous formulation which comprises surfactants of the general formula is used. In addition to water, the formulations may optionally also comprise water-miscible or at least water-dispersible organic substances or other substances. Such additives serve especially to stabilize the surfactant solution during storage or transport to the oil field. The amount of such additional solvents should, however, generally not exceed 50% by weight, preferably 20% by weight. In a particularly advantageous embodiment of the invention, exclusively water is used for formulation. Examples of water-miscible solvents include especially alcohols such as methanol, ethanol and propanol, butanol, sec-butanol, pentanol, butyl ethylene glycol, butyl diethylene glycol or butyl triethylene glycol.

In a preferred embodiment of the invention, the aqueous surfactant formulation comprises at least one anionic surfactant of the alkyl alkoxy sulfate or alkyl alkoxy sulfonated type. This is present in the aqueous surfactant formulation in a higher concentration than the cationic surfactants claimed, i.e. in a ratio of anionic surfactant to cationic surfactant of at least 5.5:4.5, preferably of at least 6:4, more preferably at least 7:3, on a molar basis, in order to guarantee that the surfactant solution remains soluble to give a clear solution owing to the charge neutralization.

In a preferred embodiment of the invention, the aqueous surfactant formulation comprises at least one anionic surfactant of the alkylarylsulfonate type. This is present in the aqueous surfactant formulation in a higher concentration than the cationic surfactants claimed, i.e. in a ratio of anionic surfactant to cationic surfactant of at least 5.5:4.5, preferably of at least 6:4, more preferably at least 7:3, on a molar basis, in order to guarantee that the surfactant solution remains soluble to give a clear solution owing to the charge neutralization.

According to the invention, the proportion of the surfactants of the general formula is not more than 49% by weight based on the proportion of all surfactants present, i.e. the surfactants of the general formula and surfactants optionally present. The proportion is preferably not more than 30% by weight.

The mixture used in accordance with the invention can preferably be used for surfactant flooding of deposits. It is especially suitable for Winsor type III microemulsion flooding (flooding in the Winsor III range or in the range of existence of the bicontinuous microemulsion phase). The technique of microemulsion flooding has already been described in detail at the outset.

In addition to the surfactants, the formulations may also comprise further components, for example C₄- to C₈ alcohols and/or basic salts (so-called “alkali surfactant flooding”). Such additives can be used, for example, to reduce retention in the formation. The ratio of the alcohols based on the total amount of surfactant used is generally at least 1:1—however, it is also possible to use a significant excess of alcohol. The amount of basic salts may typically range from 0.1% by weight to 5% by weight.

The deposits in which the process is employed generally have a temperature of at least 10° C., for example 10 to 150° C., preferably a temperature of at least 15° C. to 120° C., more preferably from 15 to 90° C. The total concentration of all surfactants together is 0.05 to 5% by weight, based on the total amount of the aqueous surfactant formulation, preferably 0.1 to 2.5% by weight. The person skilled in the art makes a suitable selection according to the desired properties, especially according to the conditions in the mineral oil formation. It is clear here to the person skilled in the art that the concentration of the surfactants can change after injection into the formation because the formulation can mix with formation water, or surfactants can also be absorbed on solid surfaces of the formation. It is the great advantage of the mixture used in accordance with the invention that the surfactants lead to a particularly good lowering of interfacial tension with simultaneous dissolution of the surfactants to give a clear solution.

It is of course possible and also advisable first to prepare a concentrate which is only diluted on site to the desired concentration for injection into the formation. In general, the total concentration of the surfactants in such a concentrate is 10 to 45% by weight.

The examples which follow are intended to illustrate the invention in detail:

### Part I: Synthesis of the Surfactants

### General Method 1: Alkoxylation by Means of KOH Catalysis

In a 2 l autoclave, the alcohol to be alkoxylated (1.0 eq) is admixed with an aqueous KOH solution which comprises 50% by weight of KOH. The amount of KOH is 0.3% by weight of the product to be prepared. While stirring, the mixture is dewatered at 100° C. and 20 mbar for 2 h. This is followed by purging three times with N₂, establishment of a feed pressure of approx. 1.3 bar of N₂ and a temperature increase to 120 to 130° C. The alkylene oxide is metered in such that the temperature remains between 125° C. and 135° C. (in the case of ethylene oxide) or 130 and 140° C. (in the case of propylene oxide). This is followed by stirring at 125 to 135° C. for a further 5 h, purging with N₂, cooling to 70° C. and emptying of the reactor. The basic crude product is neutralized with the aid of acetic acid. Alternatively, the neutralization can also be effected with commercial magnesium silicates, which are subsequently filtered off. The light-colored product is characterized with the aid of a ¹H NMR spectrum in CDCl₃, gel permeation chromatography and OH number determination, and the yield is determined.

### General Method 2: Sulfation by Means of Chlorosulfonic Acid

In a 1 l round-bottom flask, the alkyl alkoxylate to be sulfated (1.0 eq) is dissolved in 1.5-times the amount of dichloromethane (based on percent by weight) and cooled to 5 to 10° C. Thereafter, chlorosulfonic acid (1.1 eq) is added dropwise such that the temperature does not exceed 10° C. The mixture is allowed to warm up to room temperature and is stirred under an N₂ stream at this temperature for 4 h before the above reaction mixture is added dropwise to an aqueous NaOH solution of half the volume at a maximum of 15° C. The amount of NaOH is calculated to give rise to a slight excess based on the chlorosulfonic acid used. The resulting pH is approx. pH 9 to 10. The dichloromethane is removed at a maximum of 50° C. on a rotary evaporator under gentle vacuum.

The product is characterized by ¹H NMR and the water content of the solution is determined (approx. 70%).

### General Method 3: Alkoxylation of Amines

In a 2 l autoclave, the primary amine to be alkoxylated (1.0 eq) is admixed with a little water (0.1 eq). This is followed by purging three times with N₂, setting an initial pressure of approx. 1.3 bar of N₂ and increasing the temperature to 120 to 130° C. 2.0 eq of alkylene oxide are metered in such that the temperature remains between 125° C. and 135° C. This is followed by stirring at 125 to 135° C. for a further 5 h, purging with N₂, cooling to 70° C. and emptying the reactor. The basic crude product is neutralized with the aid of acetic acid. The light-colored product is characterized with the aid of a ¹H NMR spectrum in CDCl₃, gel permeation chromatography and an OH number determination and amine number, and the yield is determined.

Optionally, the amine which has been reacted with 2.0 eq of alkylene oxide can be admixed with an aqueous KOH solution which comprises 50% by weight of KOH. The amount of KOH is 0.3% by weight of the product to be prepared. The mixture is dewatered while stirring at 100° C. and 20 mbar for 2 h. This is followed by purging three times with N₂, setting an initial pressure of approx. 1.3 bar of N₂ and increasing the temperature to 120 to 130° C. The alkylene oxide is metered in such that the temperature remains between 125° C. and 135° C. (for ethylene oxide) or 130 and 140° C. (for propylene oxide). This is followed by stirring at 125 to 135° C. for a further 5 h, purging with N₂, cooling to 70° C. and emptying the reactor. The light-colored product is characterized with the aid of a ¹H NMR spectrum in CDCl₃, gel permeation chromatography and an OH number determination and amine number determination, and the yield is determined.

General Method 4: Quarternization of Amines with Dimethyl Sulfate

A 2 l glass flask is initially charged with the amine to be quaternized (1.0 eq) which is optionally diluted with the same amount of water. Then the dimethyl sulfate (1.0 eq) is slowly added dropwise while stirring, such that the temperature does not exceed 60° C. With the aid of an amine number, the conversion is determined. Stirring is continued until the degree of quaternization is 95% or more. Optionally, a small excess of dimethyl sulfate (0.1 eq) can be used. The excess dimethyl sulfate can be destroyed by briefly boiling with water. The light-colored product is characterized with the aid of a ¹H NMR spectrum in CDCl₃, gel permeation chromatography and an amine number determination, and the yield is determined.

The following alcohols and amines were used for the synthesis:

| Alcohol | Description |
|---|---|
| iC₁₇ | iso-C₁₇H₃₅—OH; oxo alcohol, prepared by |
| | hydroformylating isohexadecene, which is obtained by |
| | tetramerizing butene. The mean degree of branching of |
| | the alcohol is 3.1. |
| C₁₆C₁₈ | Commercially available fatty alcohol mixture consisting of |
| | linear C₁₆H₃₃—OH and C₁₈H₃₇—OH |
| Alkylamine | Description |
| nC6-amine | Commercially available n-hexylamine |
| nC8-amine | Commercially available n-octylamine |
| 2-EH-amine | Commercially available 2-ethylhexylamine |

### Performance Tests

The surfactants obtained were used to carry out the following tests, in order to assess the suitability thereof for tertiary mineral oil production.

### a) Solubility

An alkyl alkoxy sulfate and a cationic surfactant are dissolved at room temperature in a saline injection water or production water from a deposit (total concentration 500 to 3000 ppm), and the solution is brought to the deposit temperature. Optionally, butyl diethylene glycol (BDG) is added. After 24 h, the sample is assessed visually and used further only in the case that a clear solution is present. The injection water of the two deposits in question had salinities of 4000 to 30 000 ppm TDS (total dissolved salt). The deposit temperatures were 18° C. and 32° C. respectively.

### b) Interfacial Tension

In addition, interfacial tensions were measured directly by the spinning drop method on two dead crude oils (API of each approx. 14) and the saline original injection water at deposit temperatures of 18° C. and 32° C. For this purpose, the surfactant solution prepared in a) was used. An oil droplet was introduced into this clear solution at deposit temperature and the interfacial tension was read off after 2 h.

### Test Results

The results are shown in tables 1 to 6.

TABLE 1
Solubility in injection water at 18° C.

| | Alkyl-AO—SO₄Na | Cat. cosurfactant | BDG | Salinity | T | |
|---|---|---|---|---|---|---|
| Ex. | [900 ppm] | [100 ppm] | [ppm] | [ppm] | [° C.] | Solution |
| C1 | C₁₆C₁₈—6PO—SO₄Na | nC12-N(Me)₃⁺Cl⁻ | 400 | 12 500 | 18 | Clear |
| | | (Luviquat LS) | | | | |
| C2 | C₁₆C₁₈—6PO—SO₄Na | nC12-N(Me)₃⁺Cl⁻ | 0 | 12 500 | 18 | Clear |
| | | (Luviquat LS) | | | | |
| C3 | C₁₆C₁₈—6PO—SO₄Na | — | 0 | 12 500 | 18 | Clear |
| C4 | C₁₆C₁₈—7PO—0.1EO—SO₄Na | nC12-N(Me)₃⁺Cl⁻ | 0 | 12 500 | 18 | Clear |
| | | (Luviquat LS) | | | | |
| C5 | iC₁₇—7PO—0.1EO—SO₄Na | nC12-N(Me)₃⁺Cl⁻ | 0 | 12 500 | 18 | Clear |
| | | (Luviquat LS) | | | | |
| 6 | C₁₆C₁₈—6PO—SO₄Na | nC8-N(EO)²Me⁺MeOSO₃⁻ | 200 | 12 500 | 18 | Clear |
| 7 | C₁₆C₁₈—6PO—SO₄Na | nC8-N(EO)²Me⁺MeOSO₃⁻ | 200 | 24 300 | 18 | Clear |

As can be seen in table 1, there were some combinations which lead to a clear surfactant formulation under the given conditions.

TABLE 2
Measurements on crude oil I injection water at 18° C.

| | Alkyl-AO—SO₄Na | Cat. cosurfactant | BDG | Salinity | T | IFT |
|---|---|---|---|---|---|---|
| Ex. | [900 ppm] | [100 ppm] | [ppm] | [ppm] | [° C.] | [mN/m] |
| C1 | C₁₆C₁₈—6PO-SO₄Na | nC12-N(Me)₃⁺Cl⁻ | 400 | 12 500 | 18 | 0.217 |
| | | (Luviquat LS) | | | | |
| C2 | C₁₆C₁₈—6PO-SO₄Na | nC12-N(Me)₃⁺Cl⁻ | 0 | 12 500 | 18 | 0.0963 |
| | | (Luviquat LS) | | | | |
| C3 | C₁₆C₁₈—6PO-SO₄Na | — | 0 | 12 500 | 18 | 0.11 |
| C4 | C₁₆C₁₈—7PO-0.1EO-SO₄Na | nC12-N(Me)₃⁺Cl⁻ | 0 | 12 500 | 18 | 0.153 |
| | | (Luviquat LS) | | | | |
| C5 | iC₁₇—7PO-0.1EO-SO₄Na | nC12-N(Me)₃⁺Cl⁻ | 0 | 12 500 | 18 | 0.0825 |
| | | (Luviquat LS) | | | | |
| 6 | C₁₆C₁₈—6PO-SO₄Na | nC8-N(EO)₂Me⁺MeOSO₃⁻ | 200 | 12 500 | 18 | 0.0083 |
| 7 | C₁₆C₁₈—6PO-SO₄Na | nC8-N(EO)₂Me⁺MeOSO₃⁻ | 200 | 24 300 | 18 | 0.0028 |

On comparison with table 2, however, it is noticeable that the cationic surfactants based on a radical having 12 carbon atoms give significantly poorer interfacial tensions than in Example 6. This is surprising since surfactants with a relatively long alkyl radical typically give better interfacial tensions. As can be seen in Example 7, the surfactants claimed give interfacial tensions of <0.01 mN/m not only in the case of low salinities (Example 6, total salt content 12 500 ppm) but also at higher salinities of total salt content of about 24 300 ppm.

TABLE 3
Solubility in injection water at 18° C.

| Ex. | Alkyl-AO—SO₄Na [800 ppm] | Cat. cosurfactant [200 ppm] | BDG [ppm] | Salinity [ppm] | T [° C.] | Solution |
|---|---|---|---|---|---|---|
| 1 | C₁₆C₁₈—8PO—SO₄Na | nC8-N(EO)₅⁺MeOSO₃⁻ | 200 | 12 500 | 18 | Clear |
| 2 | C₁₆C₁₈—8PO—SO₄Na | nC8-N(EO)₅⁺MeOSO₃⁻ | 200 | 16 100 | 18 | Clear |
| 3 | C₁₆C₁₈—8PO—SO₄Na | nC8-N(EO)₅⁺MeOSO₃⁻ | 200 | 24 300 | 18 | Clear |

In table 3, no solubility problems whatsoever occur even with the surfactant formulations which comprise comparatively highly ethoxylated cationic surfactants.

TABLE 4
Measurements on crude oil I and injection water at 18° C.

| Ex. | Alkyl-AO—SO₄Na [800 ppm] | Cat. cosurfactant [200 ppm] | BDG [ppm] | Salinity [ppm] | T [° C.] | IFT [mN/m] |
|---|---|---|---|---|---|---|
| 1 | C₁₆C₁₈—8PO—SO₄Na | nC8-N(EO)₅⁺MeOSO₃⁻ | 200 | 12 500 | 18 | 0.0078 |
| 2 | C₁₆C₁₈—8PO—SO₄Na | nC8-N(EO)₅⁺MeOSO₃⁻ | 200 | 16 100 | 18 | 0.0065 |
| 3 | C₁₆C₁₈—8PO—SO₄Na | nC8-N(EO)₅⁺MeOSO₃⁻ | 200 | 24 300 | 18 | 0.0021 |

As can be seen in table 4, the combination of alkyl alkoxy sulfate and comparatively highly ethoxylated cationic surfactant has an interfacial tension of <0.01 mN/m over a wide salinity range.

TABLE 5
Solubility in injection water at 32° C.

| | Alkyl-AO—SO₄Na | Cat. cosurfactant | NaOH | BDG | Salinity | T | |
|---|---|---|---|---|---|---|---|
| Ex. | [800 ppm] | [200 ppm] | [ppm] | [ppm] | [ppm] | [° C.] | Solution |
| C1 | C₁₆C₁₈—6PO—SO₄Na | nC12-N(EO)₂⁺MeOSO₃⁻ | 2000 | 500 | 13 500 | 32 | Clear |
| 2 | C₁₆C₁₈—6PO—SO₄Na | nC8-N(EO)₂⁺MeOSO₃⁻ | 2000 | 500 | 13 500 | 32 | Clear |
| 3 | C₁₆C₁₈—6PO—SO₄Na | 2-EH-N-(EO)₂Me⁺MeOSO₃⁻ | 2000 | 500 | 13 500 | 32 | Clear |
| 4 | C₁₆C₁₈—6PO—SO₄Na | nC6-N(EO)₂Me⁺MeOSO₃⁻ | 2000 | 500 | 13 500 | 32 | Clear |

In table 5, the solubility of the surfactant formulations was considered for a deposit with a higher temperature (32° C. instead of 18° C.). In addition to the tests so far, the formulations comprise a base in the form of NaOH.

TABLE 6
Measurements on crude oil II injection water at 32° C.

| | Alkyl-AO—SO₄Na | Cat. cosurfactant | NaOH | BDG | Salinity | T | IFT |
|---|---|---|---|---|---|---|---|
| Ex. | [800 ppm] | [200 ppm] | [ppm] | [ppm] | [ppm] | [° C.] | [mN/m] |
| C1 | C₁₆C₁₈—6PO—SO₄Na | nC12-N(EO)₂⁺MeOSO₃⁻ | 2000 | 500 | 13 500 | 32 | 0.3275 |
| 2 | C₁₆C₁₈—6PO—SO₄Na | nC8-N(EO)₂⁺MeOSO₃⁻ | 2000 | 500 | 13 500 | 32 | 0.0168 |
| 3 | C₁₆C₁₈—6PO—SO₄Na | 2-EH-N-(EO)₂Me⁺MeOSO₃⁻ | 2000 | 500 | 13 500 | 32 | 0.0202 |
| 4 | C₁₆C₁₈—6PO—SO₄Na | nC6-N(EO)₂Me⁺MeOSO₃⁻ | 2000 | 500 | 13 500 | 32 | 0.0159 |

As can be seen in table 6, it is possible with the aid of the cationic cosurfactants which possess an alkyl radical having 6-10 carbon atoms to achieve low interfacial tensions of 0.02 mN/m or less (Examples 2 to 4). The comparatively long-chain cationic surfactant in Comparative Example C1 again exhibits an interfacial tension which is one order of magnitude higher.

1. A surfactant of the general formula
R¹N⁺(R²)ₘ(R³)ₙ(R⁴)X⁻ where
R¹ is a linear or branched, saturated or unsaturated, aliphatic and/or aromatic hydrocarbyl radical having 6 to 10 carbon atoms,
R² and R³ are each independently methyl, ethyl and benzyl, or ethyleneoxy, propyleneoxy and/or butyleneoxy and/or pentyleneoxy,
R⁴ is an alkyl group or hydroxyalkyl group having 1 to 4 carbon atoms, a benzyl group, or a phenyl-CH₂—CH₂— or a phenyl-CH(CH₃)-group,
m is from 1 to 8, and
n is from 1 to 8, where the sum of m+n is in the range from 2 to 8, and
X is an anion.

2. The surfactant according to claim 1, wherein R² and R³ are each independently methyl, ethyl or benzyl, and n=m=1.

3. The surfactant according to claim 1, wherein R² and R³ are each ethyleneoxy and R⁴ is methyl or ethyl.

4. The surfactant according to claim 1, wherein the sum of n+m is in the range from 2 to 5.

5. A surfactant formulation comprising at least one surfactant of the general formula
R¹N⁺(R²)ₘ(R³)ₙ(R⁴)X⁻ where
R¹ is a linear or branched, saturated or unsaturated, aliphatic and/or aromatic hydrocarbyl radical having 6 to 10 carbon atoms,
R² and R³ are each independently methyl, ethyl and benzyl, or ethyleneoxy, propyleneoxy and/or butyleneoxy and/or pentyleneoxy,
R⁴ is an alkyl group or hydroxyalkyl group having 1 to 4 carbon atoms, a benzyl group, or a phenyl-CH₂—CH₂— or a phenyl-CH(CH₃)-group,
m is from 1 to 8, and
n is from 1 to 8, where the sum of m+n is in the range from 2 to 8, and
X is an anion.

6. The surfactant formulation according to claim 5, wherein R² and R³ are each independently methyl, ethyl or benzyl, and n=m=1.

7. The surfactant formulation according to claim 5, wherein the concentration of all surfactants together is 0.05 to 5% by weight, based on the total amount of the aqueous surfactant formulation.

8. A process for mineral oil production by means of Winsor type III microemulsion flooding, in which an aqueous surfactant formulation comprising at least one ionic surfactant, for the purpose of lowering the interfacial tension between oil and water to <0.1 mN/m, is injected through at least one injection borehole into a mineral oil deposit, and crude oil is withdrawn from the deposit through at least one production borehole, wherein the surfactant formulation comprises at least one surfactant of the general formula
R¹N⁺(R²)ₘ(R³)ₙ(R⁴)X⁻ where
R¹ is a linear or branched, saturated or unsaturated, aliphatic and/or aromatic hydrocarbyl radical having 6 to 10 carbon atoms,
R² and R³ are each independently methyl, ethyl and benzyl, or ethyleneoxy, propyleneoxy and/or butyleneoxy and/or pentyleneoxy,
R⁴ is an alkyl group or hydroxyalkyl group having 1 to 4 carbon atoms, a benzyl group, or a phenyl-CH₂—CH₂— or a phenyl-CH(CH₃)-group,
m is from 1 to 8, and
n is from 1 to 8, where the sum of m+n is in the range from 2 to 8, and
X is an anion.

9. The process according to claim 8, wherein the sum of m+n is in the range from 2 to 5.

10. The process for mineral oil production according to claim 9, wherein R² and R³ are each independently methyl, ethyl or benzyl, and n=m=1.

11. The process according to claim 8, wherein the aqueous surfactant formulation comprises at least one anionic surfactant of the alkyl alkoxy sulfate or alkyl alkoxy sulfonated type, which is present in the aqueous surfactant formulation in greater amounts than the cationic surfactants claimed.

12. The process according to claim 8, wherein the aqueous surfactant formulation also comprises an anionic surfactant of the alkylarylsulfonate type, which is present in the aqueous surfactant formulation in greater amounts than the cationic surfactants claimed.

13. The process according to claim 8, wherein the concentration of all surfactants together is 0.05 to 5% by weight, based on the total amount of the aqueous surfactant formulation.
